(19) United States
(12) Patent Application Publication
(10) Pub. No.: US 2023/0100367 A1
(43) Pub. Date: Mar. 30, 2023

(54) METHODS OF TREATING CDGSH IRON SULFUR DOMAIN 2 INSUFFICIENCY-ASSOCIATED DISORDERS

(71) Applicants: National Yang Ming Chiao Tung University, Taipei (TW); National Health Research Institutes, Miaoli County (TW); Chang Gung Medical Foundation, Chang Gung Memorial Hospital, Linkou, Taoyuan City (TW)

(72) Inventors: Ting-Fen Tsai, Taipei (TW); Jinq-Chyi Lee, Miaoli County (TW); Hui-Yi Shiao, Miaoli County (TW); Chi-Hsiao Yeh, Taoyuan City (TW)

(21) Appl. No.: 17/894,333

(22) Filed: Aug. 24, 2022

Related U.S. Application Data
(60) Provisional application No. 63/236,566, filed on Aug. 24, 2021.

(51) Int. Cl. C07D 333/36; C07D 417/12; A61P 9/00

(52) U.S. Cl. CPC C07D 333/36; C07D 417/12; A61P 9/00

(57) ABSTRACT

A method of treating a Cisd2 insufficiency-associated disorder. The method includes administering to a subject in need a compound of formula (I). Also disclosed is a method of increasing Cisd2 gene expression.

### BACKGROUND

CDGSH iron sulfur domain 2 (Cisd2) is an iron-sulfur transmembrane protein encoded by the Cisd2 gene. This protein localizes on the mitochondrial outer membrane, the endoplasmic reticulum (ER) membrane, and other mitochondria or ER associated membranes.

Cisd2 is a fundamental regulator of aging and metabolism via the maintenance of cytosolic Ca²⁺ homeostasis, ER integrity, and mitochondrial function. See Wu et al., Human Molecular Genetics 21, 3956-68 (2012).

Cisd2 insufficiency results in degeneration and pathogenesis of multiple tissues and organs, e.g., brain, heart, liver, skeletal muscle, skin, white adipose tissue, and beta-cells of pancreas.

Upregulation of Cisd2 has been reported to attenuate Aβ-mediated neuron loss in Alzheimer's disease and ameliorate various aging-related disorders, including cardiac dysfunction, fatty liver disease and inflammation, muscle degeneration and sarcopenia, skin aging, glucose intolerance, diabetes, and hypertensive cardiomyopathy.

Cisd2 activators are potential therapies to the above-mentioned disorders. Nevertheless, it is a challenge to develop an effective and safe Cisd2 activator as there are not many publications to provide guidance. Indeed, the US Food and Drug Administration has not yet approved any Cisd2 activator for medical use.

There is a need to develop Cisd2 activators as effective therapies to treat disorders associated with Cisd2 insufficiency.

### SUMMARY

It was discovered unexpectedly that certain thiophene compounds are effective in treating a subject suffering from a disorder associated with Cisd2 insufficiency by increasing the Cisd2 level.

Accordingly, one aspect of this invention relates to a method of treating a Cisd2 insufficiency-associated disorder. The method contains the steps of (i) identifying a subject suffering from a Cisd2 insufficiency-associated disorder and (ii) administering to the subject an effective amount of a thiophene compound of formula (I):

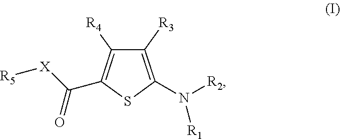

in which, R₁ is H or C₁₋₆ alkyl; R₂ is H, C₁₋₆ alkyl, or COR_{b}, R_{b} being C₁₋₆ alkyl; R₃ is CN or CORₐ, Rₐ being OH, NH₂, NHCH₂CN, C₁₋₆ alkyl, or C₁₋₆ alkoxy; R₄ is H, halo, C₁₋₆ alkyl, or C₁₋₆ alkoxy; R₅ is aryl, heteroaryl, or C₁₋₆ alkyl; and X is O, NH, or CH₂.

The method is suitable for treating the following Cisd2 insufficiency-associated disorders: non-alcoholic fatty liver disease, non-alcoholic steatohepatitis, chemotherapy-induced cardiotoxicity, chemotherapy-induced cachexia, hepatotoxicity, aging-related arrhythmogenesis, an aging-related cardiac pathological change, and hypertensive cardiomyopathy.

Preferred compounds have one or more of the following features: R₁ is H; R₂ is H or C(O)CH₃; R₃ is CN, CO₂CH₃, CO₂CH₂CH₃, CO₂C(CH₃)₃, or COCH₃; R₄ is methyl; and R₅ is aryl or heteroaryl. Exemplary R₅ moieties include:

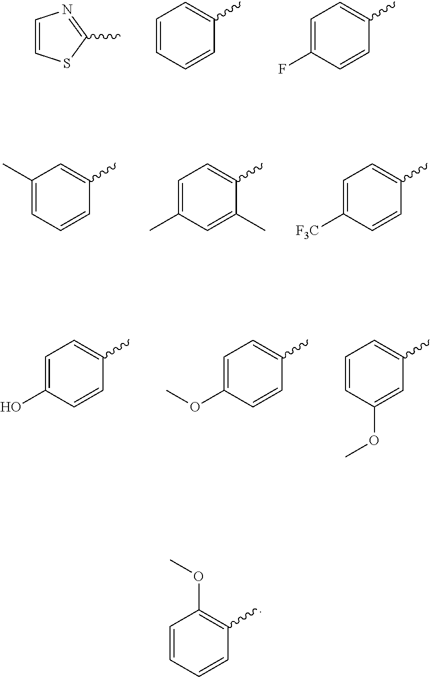

Table 1 below lists 35 exemplary thiophene compounds including their structures that are useful for the methods of this invention.

TABLE 1

| No. | Chemical Structure |
|---|---|
| 1 | 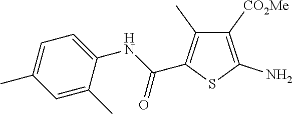 [SMILES: COC(=O)c1c(N)sc(C(=O)Nc2ccc(C)cc2C)c1C] |
| 2 | 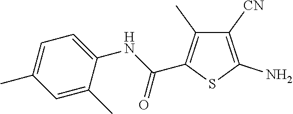 [SMILES: Cc1ccc(NC(=O)c2sc(N)c(C#N)c2C)c(C)c1] |
| 3 | 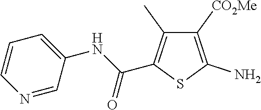 [SMILES: COC(=O)c1c(N)sc(C(=O)Nc2cccnc2)c1C] |
| 4 | 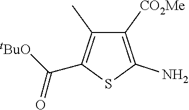 [SMILES: COC(=O)c1c(N)sc(C(=O)OC(C)(C)C)c1C] |
| 5 | 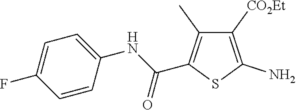 [SMILES: CCOC(=O)c1c(N)sc(C(=O)Nc2ccc(F)cc2)c1C] |
| 6 | 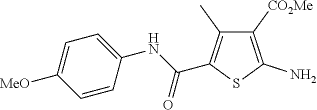 [SMILES: COC(=O)c1c(N)sc(C(=O)Nc2ccc(OC)cc2)c1C] |
| 7 | 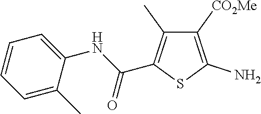 [SMILES: COC(=O)c1c(N)sc(C(=O)Nc2ccccc2C)c1C] |
| 8 | 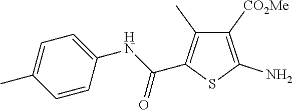 [SMILES: COC(=O)c1c(N)sc(C(=O)Nc2ccc(C)cc2)c1C] |
| 9 | 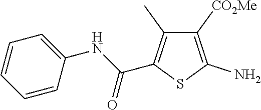 [SMILES: COC(=O)c1c(N)sc(C(=O)Nc2ccccc2)c1C] |
| 10 | 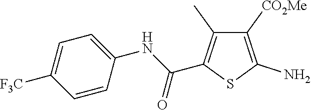 [SMILES: COC(=O)c1c(N)sc(C(=O)Nc2ccc(C(F)(F)F)cc2)c1C] |
| 11 | 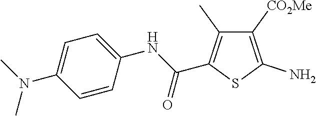 [SMILES: COC(=O)c1c(N)sc(C(=O)Nc2ccc(N(C)C)cc2)c1C] |
| 12 | 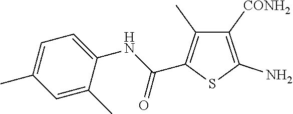 [SMILES: Cc1ccc(NC(=O)c2sc(N)c(C(N)=O)c2C)c(C)c1] |
| 13 | 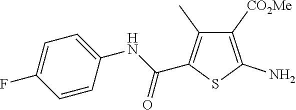 [SMILES: COC(=O)c1c(N)sc(C(=O)Nc2ccc(F)cc2)c1C] |
| 14 | 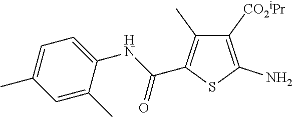 |
| 15 | 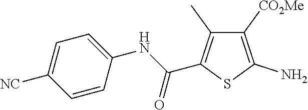 [SMILES: COC(=O)c1c(N)sc(C(=O)Nc2ccc(C#N)cc2)c1C] |
| 16 | 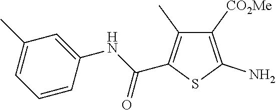 [SMILES: COC(=O)c1c(N)sc(C(=O)Nc2cccc(C)c2)c1C] |
| 17 | 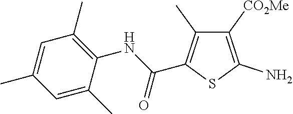 [SMILES: COC(=O)c1c(N)sc(C(=O)Nc2c(C)cc(C)cc2C)c1C] |
| 18 | 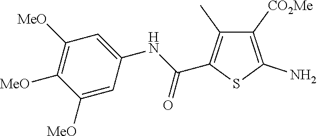 [SMILES: COC(=O)c1c(N)sc(C(=O)Nc2cc(OC)c(OC)c(OC)c2)c1C] |
| 19 | 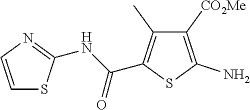 [SMILES: COC(=O)c1c(N)sc(C(=O)Nc2nccs2)c1C] |
| 20 | 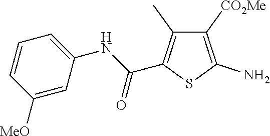 [SMILES: COC(=O)c1c(N)sc(C(=O)Nc2cccc(OC)c2)c1C] |
| 21 | 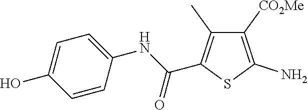 [SMILES: COC(=O)c1c(N)sc(C(=O)Nc2ccc(O)cc2)c1C] |
| 22 | 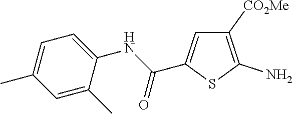 [SMILES: COC(=O)c1cc(C(=O)Nc2ccc(C)cc2C)sc1N] |
| 23 | 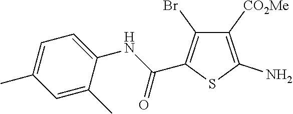 [SMILES: COC(=O)c1c(N)sc(C(=O)Nc2ccc(C)cc2C)c1Br] |
| 24 | 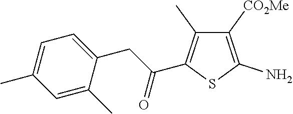 [SMILES: COC(=O)c1c(N)sc(C(=O)Cc2ccc(C)cc2C)c1C] |
| 25 | 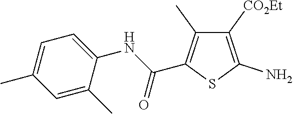 [SMILES: CCOC(=O)c1c(N)sc(C(=O)Nc2ccc(C)cc2C)c1C] |
| 26 | 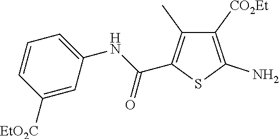 [SMILES: CCOC(=O)c1cccc(NC(=O)c2sc(N)c(C(=O)OCC)c2C)c1] |
| 27 | 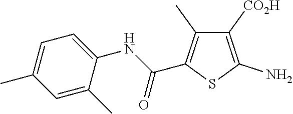 [SMILES: Cc1ccc(NC(=O)c2sc(N)c(C(=O)O)c2C)c(C)c1] |
| 28 | 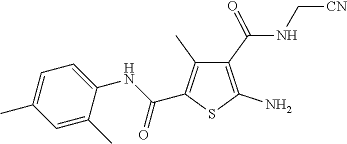 [SMILES: Cc1ccc(NC(=O)c2sc(N)c(C(=O)NCC#N)c2C)c(C)c1] |
| 29 | 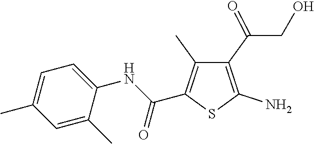 [SMILES: Cc1ccc(NC(=O)c2sc(N)c(C(=O)CO)c2C)c(C)c1] |
| 30 | 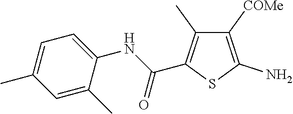 |
| 31 | 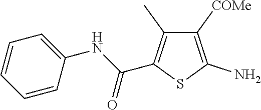 |
| 32 | 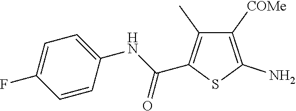 |
| 33 | 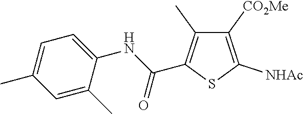 [SMILES: COC(=O)c1c(NC(C)=O)sc(C(=O)Nc2ccc(C)cc2C)c1C] |
| 34 | 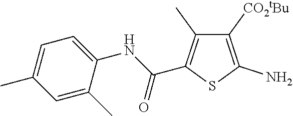 [SMILES: Cc1ccc(NC(=O)c2sc(N)c(C(=O)OC(C)(C)C)c2C)c(C)c1] |
| 35 | 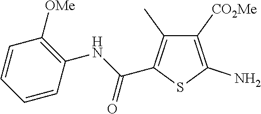 [SMILES: COC(=O)c1c(N)sc(C(=O)Nc2ccccc2OC)c1C] |

Among Compounds 1-35 above, more preferred compounds are Compounds 1, 5, 6, 9, 10, 13, 16, 19, 20, 21, 25, 30, and 32.

Another aspect of this invention relates to a method of increasing Cisd2 gene expression, containing the steps of (i) identifying a subject in need of increase in Cisd2 gene expression and (ii) administering to the subject an effective amount of a compound of formula (I) as described above.

Also within the scope of this invention is a pharmaceutical composition containing any of the compounds described above and a pharmaceutically acceptable carrier. The pharmaceutical composition is useful for treating a Cisd2 insufficiency-associated disorder or increasing Cisd2 gene expression.

The term “alkyl” herein refers to a straight or branched hydrocarbon group, containing 1-20 (e.g., 1-10 and 1-6) carbon atoms. Examples include methyl, ethyl, n-propyl, i-propyl, n-butyl, i-butyl, and t-butyl. Alkyl includes its halo substituted derivatives, i.e., haloalkyl, which refers to alkyl substituted with one or more halogen (chloro, fluoro, bromo, or iodo) atoms. Examples include trifluoromethyl, bromomethyl, and 4,4,4-trifluorobutyl. The term “alkoxy” refers to an —O— alkyl group. Examples include methoxy, ethoxy, propoxy, and isopropoxy. Alkoxy includes haloalkoxy, referring to alkoxy substituted with one or more halogen atoms. Examples include —O—CH₂C₁ and —O—CHClCH₂Cl.

The term “aryl” refers to a 6-carbon monocyclic, 10-carbon bicyclic, 14-carbon tricyclic aromatic ring system wherein each ring may have 1 to 5 substituents. Examples of aryl groups include phenyl, naphthyl, and anthracenyl.

The term “heteroaryl” refers to an aromatic 5-8 membered monocyclic, 8-12 membered bicyclic, or 11-14 membered tricyclic ring system having one or more heteroatoms (e.g., O, N, P, and S). Examples include triazolyl, oxazolyl, thiadiazolyl, tetrazolyl, pyrazolyl, pyridyl, furyl, imidazolyl, benzimidazolyl, pyrimidinyl, thienyl, quinolinyl, indolyl, thiazolyl, and benzothiazolyl.

The term “halo” refers to a fluoro, chloro, bromo, or iodo radical.

Alkyl, aryl, heteroaryl, and alkoxy mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cycloalkyl, and heterocycloalkyl, in which alkyl, alkenyl, alkynyl, alkyloxy, aryl, heteroaryl cycloalkyl, and heterocycloalkyl may further substituted.

The term “compound”, when referring to a compound of Formula (I), also covers its salts, solvates, and prodrugs. A salt can be formed between an anion and a positively charged group (e.g., amino) on a compound; examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. A salt can also be formed between a cation and a negatively charged group. Examples of a suitable cation include sodium ion, potassium ion, magnesium ion, calcium ion, and an ammonium cation such as tetramethylammonium ion. Further, a salt can contain quaternary nitrogen atoms. A solvate refers to a complex formed between an active compound and a pharmaceutically acceptable solvent. Examples of a pharmaceutically acceptable solvent include water, ethanol, isopropanol, ethyl acetate (“EtOAc”), acetic acid, and ethanolamine. A prodrug refers to a compound that, after administration, is metabolized into a pharmaceutically active drug. Examples of a prodrug include esters and other pharmaceutically acceptable derivatives, which, upon administering to a subject, are capable of providing active compounds of this invention.

The details of several embodiments of the present invention are set forth in both the description and the examples below. All features, objects, and advantages of the invention will be apparent from the description and claims.

### DETAILED DESCRIPTION

The invention is based on surprising discovery of certain thiophene compounds that are effective in increasing Cisd2 gene expression and thus useful to treat Cisd2 insufficiency-associated disorders.

The thiophene compounds are represented by formula (I) as described above. Their syntheses are achieved by applying well known methods in the art. See, e.g., R. Larock, Comprehensive Organic Transformations (3^{rd} Ed., John Wiley and Sons 2018); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); and L. Paquette, ed., Encyclopedia of Reagents for Organic Synthesis (2^{nd} ed., John Wiley and Sons 2009) and subsequent editions thereof.

Preparation of certain thiophene compounds is described in Example 1 below, as well as International Application Publication WO 2005/033102.

The compounds thus prepared can be purified following conventional methods such as crystallization, distillation/vacuum distillation, flash chromatography over silica, and preparative liquid chromatography.

Some thiophene compounds useful in this invention contain a non-aromatic double bond or one or more asymmetric centers. Each of them occurs as a racemate or a racemic mixture, a single R enantiomer, a single S enantiomer, an individual diastereomer, a diastereometric mixture, a cis-isomer, or a trans-isomer. Compounds of such isomeric forms are within the scope of this invention. They can be present as a mixture or can be isolated using chiral synthesis or chiral separation technologies.

The thiophene compounds of formula (I) can be initially screened using an in vitro method to identify Cisd2 activation activity.

A typical in vitro screening method includes the following steps: (i) obtaining an initial level of Cisd2 in a batch of human embryonic kidney cells 293 (HEK-293 cells) that express Cisd2, (ii) treating the batch of HEK-293 cells with a compound of this invention, and (iii) analyzing the level of Cisd2 after the treatment, thereby determining the potency of the compound as a Cisd2 activator.

The compounds of formula (I) are effective Cisd2 activators as shown in below examples. They are useful in treating Cisd2 insufficiency-associated disorders, such as liver diseases, metabolic diseases, heart diseases, cachexia, and aging-associated diseases.

A compound of formula (I) is preferably formulated into a pharmaceutical composition containing a pharmaceutical carrier. The pharmaceutical composition is then given to a subject in need thereof to treat a Cisd2 insufficiency-associated disorder or increase the Cisd2 gene expression.

To practice the method of the present invention, a composition having one or more of the above-described thiophene compounds can be administered parenterally, orally, nasally, rectally, topically, or buccally.

The term “parenteral” as used herein encompasses subcutaneous, intracutaneous, intravenous, intraperitoneal, intramuscular, intraarticular, intraarterial, intrasynovial, intrasternal, intrathecal, intralesional, and intracranial injection of a sterile injectable composition. Indeed, the term refers to any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or di-glycerides). Fatty acids, such as oleic acid and its glyceride derivatives, are useful in the preparation of injectables, as are natural pharmaceutically acceptable oils, such as olive oil and castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens and Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Oral solid dosage forms can be prepared by spray dried techniques; hot melt extrusion strategy, micronization, and nano milling technologies.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having an active compound can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples of other carriers include colloidal silicon oxide, magnesium stearate, cellulose, sodium lauryl sulfate, and D&C Yellow #10.

This invention also features use of one or more of the compounds of formula (I) for the manufacture of a medicament or for treating and preventing Cisd2 insufficiency-associated disorders or increasing the Cisd2 gene expression.

The term “treating” refers to application or administration of the compound to a subject with the purpose to cure, alleviate, relieve, alter, remedy, improve, or affect the disease, the symptom, or the predisposition. “An effective amount” refers to the amount of the compound which is required to confer the desired effect on the subject. Effective amounts vary, as recognized by those skilled in the art, depending on route of administration, excipient usage, and the possibility of co-usage with other therapeutic treatments such as use of other active agents.

Without further elaboration, it is believed that one skilled in the art can, based on the above description, utilize the present invention to its fullest extent. The following examples are to be construed as merely illustrative and not limitative of the remainder of the disclosure in any way whatsoever. All publications cited herein are hereby incorporated by reference in their entirety.

Set forth below are examples illustrating preparation and efficacy evaluation of thiophene compounds of formula (I).

### EXAMPLES

Compounds 1-35 were prepared as illustrated in Example 1 below. Their efficacy was evaluated by in vivo and in vitro studies described in Examples 2-9.

### EXAMPLE 1: Preparation of Compounds 1-35

Compounds 1-20, 22, 25, 26, 34, and 35 each were prepared following a procedure similar to that described in Meng et al., J. Med. Chem. 58, 8166-81 (2015).

Methyl 2-amino-5-[(2,4-dimethylphenyl)carbamoyl]-4-methylthiophene carboxylate (Compound 1): ¹H NMR (300 MHz, d6-DMSO) δ 9.04 (s, 1H), 7.71 (s, 2H), 7.20 (d, 1H), 7.03 (s, 1H), 6.97 (d, 1H), 3.74 (s, 3H), 2.53 (s, 3H), 2.26 (s, 3H), 2.15 (s, 3H); LC-MS (ESI) m/z: 319.1 [M+H]⁺.

5-Amino-4-cyano-N-(2,4-dimethylphenyl)-3-methylthiophene-2-carboxamide (Compound 2): ¹H NMR (300 MHz, d6-DMSO) δ 8.99 (s, 1H), 7.69 (s, 2H), 7.20 (d, 1H), 7.04 (s, 1H), 6.98 (d, 1H), 2.38 (s, 3H), 2.26 (s, 3H), 2.14 (s, 3H); LC-MS (ESI) m/z: 286.1 [M+H]⁺.

Methyl 2-amino-4-methyl-5-(pyridin-3-ylcarbamoyl)thiophene-3-carboxylate (Compound 3): ¹H NMR (400 MHz, CD₃OD) δ 8.75 (d, 1H), 8.27 (dd, 1H), 8.10 (ddd, 1H), 7.41 (dd, 1H), 3.83 (s, 3H), 2.59 (s, 3H); LC-MS (ESI) m/z: 292.1 [M+H]+.

2-tert-Butyl 4-methyl 5-amino-3-methylthiophene-2,4-dicarboxylate (Compound 4): ¹H NMR (300 MHz, CDCl₃) δ 6.43 (brs, 2H), 3.84 (s, 3H), 2.66 (s, 3H), 1.53 (s, 9H); LC-MS (ESI) m/z: 294.1 [M+Na]+.

Ethyl 2-amino-5-[(4-fluorophenyl)carbamoyl]-4-methylthiophene-3-carboxylate (Compound 5): ¹H NMR (400 MHz, CDCl₃) δ 7.51-7,45 (m, 2H), 7.25 (brs, 1H), 7.07-7.01 (m, 2H), 6.43 (brs, 2H), 4.33 (q, 2H), 2.68 (s, 3H), 1.38 (t, 3H); LC-MS (ESI) m/z: 323.1 [M+H]⁺.

Methyl 2-amino-5-[(4-methoxyphenyl)carbamoyl]-4-methylthiophene-3-carboxylate (Compound 6): ¹H NMR (300 MHz, CD₃OD) δ 7.46-7.41 (m, 2H), 6.92-6.87 (m, 2H), 3.82 (s, 3H), 3.78 (s, 3H), 2.56 (s, 3H); LC-MS (ESI) m/z: 321.2 [M+H]⁺.

Methyl 2-amino-4-methyl-5-[(2-methylphenyl)carbamoyl]thiophene-3-carboxylate (Compound 7): ¹H NMR (400 MHz, CDCl₃) δ 7.93 (d, 1H), 7.26-7.20 (m, 3H), 7.08 (ddd, 1H), 6.42 (s, 2H), 3.87 (s, 3H), 2.70 (s, 3H), 2.31 (s, 3H); LC-MS (ESI) m/z: 305.2 [M+H]⁺.

Methyl 2-amino-4-methyl-5-[(4-methylphenyl)carbamoyl]thiophene-3-carboxylate (Compound 8): ¹H NMR (300 MHz, CDCl₃) δ 7.42-7.39 (m, 2H), 7.25 (brs, 1H), 7.14 (d, 2H), 6.42 (brs, 2H), 3.86 (s, 3H), 2.66 (s, 3H), 2.33 (s, 3H); LC-MS (ESI) m/z: 305.3 [M+H]⁺.

Methyl 2-amino-4-methyl-5-(phenylcarbamoyl)thiophene-3-carboxylate (Compound 9): ¹H NMR (400 MHz, CDCl₃) δ 7.53 (d, 2H), 7.35 (dd, 2H), 7.28 (brs, 1H), 7.13 (dd, 1H), 6.42 (brs, 2H), 3.86 (s, 3H), 2.68 (s, 3H); LC-MS (ESI) m/z: 291.2 [M+H]⁺.

Methyl 2-amino-4-methyl-5-{-[4-(trifluoromethyl)phenyl]carbamoyl}thiophene-3-carboxylate (Compound 10): ¹H NMR (400 MHz, CD₃OD) δ 7.77 (d, 2H), 7.61 (d, 2H), 3.83 (s, 3H), 2.58 (s, 3H); LC-MS (ESI) m/z: 359.2 [M+H]⁺.

Methyl 2-amino-5-{-[4-(dimethylamino)phenyl]carbamoyl}-4-methylthiophene-3-carboxylate (Compound 11): ¹H NMR (400 MHz, d6-DMSO) δ 9.40 (s, 1H), 7.70 (s, 2H), 7.40 (d, 2H), 6.68 (d, 2H), 3.73 (s, 3H), 2.84 (s, 6H), 2.46 (s, 3H); LC-MS (ESI) m/z: 334.3 [M+H]⁺.

5-Amino-N²-(2,4-dimethylphenyl)-3-methylthiophene-2,4-dicarboxamide (Compound 12): ¹H NMR (400 MHz, d6-Acetone) δ 8.06 (brs, 1H), 7.46 (d, 1H), 7.29 (brs, 2H), 7.03 (s, 1H), 6.98 (d, 1H), 6.43 (brs, 2H), 2.71 (s, 3H), 2.27 (s, 3H), 2.25 (s, 3H); LC-MS (ESI) m/z: 304.3 [M+H]⁺.

Methyl 2-amino-5-[(4-fluorophenyl)carbamoyl]-4-methylthiophene-3-carboxylate (Compound 13): ¹H NMR (400 MHz, d6-DMSO): δ 9.74 (s, 1H), 7.77 (s, 2H), 7.62-7.59 (m, 2H), 7.17-7.12 (m, 2H), 3.74 (s, 3H), 2.48 (s, 3H); LC-MS (ESI) m/z: 309.2 [M+H]⁺.

Propan-2-yl 2-amino-5-[(2,4-dimethylphenyl)carbamoyl]-4-methylthiophene-3-carboxylate (Compound 14): ¹H NMR (400 MHz, CDCl₃) δ 7.72 (d, 1H), 7.14 (s, 1H), 7.04-7.02 (m, 2H), 6.47 (s, 2H), 5.22 (sept, 1H), 2.69 (s, 3H), 2.30 (s, 3H), 2.26 (s, 3H), 1.36 (d, 6H); LC-MS (ESI) m/z: 347.3 [M+H]⁺.

Methyl 2-amino-5-[(4-cyanophenyl)carbamoyl]-4-methylthiophene-3-carboxylate (Compound 15): ¹H NMR (400 MHz, CD₃OD) δ 7.78-7.76 (m, 2H), 7.68-7.66 (m, 2H), 3.82 (s, 3H), 2.57 (s, 3H); LC-MS (ESI) m/z: 316.2 [M+H]⁺.

Methyl 2-amino-4-methyl-5-[(3-methylphenyl)carbamoyl]thiophene-3-carboxylate (Compound 16): ¹H NMR (300 MHz, CD₃OD) δ 7.37-7.33 (m, 2H), 7.20 (dd, 1H), 6.94 (d, 1H), 3.82 (s, 3H), 2.56 (s, 3H), 2.33 (s, 3H); LC-MS (ESI) m/z: 305.2 [M+H]⁺.

Methyl 2-amino-4-methyl-5-[(2,4,6-trimethylphenyl)carbamoyl]thiophene carboxylate (Compound 17): ¹H NMR (300 MHz, CDCl₃) δ 6.90 (s, 2H), 6.75 (brs, 1H), 6.39 (brs, 2H), 3.85 (s, 3H), 2.67 (s, 3H), 2.27 (s, 3H), 2.22 (s, 6H); LC-MS (ESI) m/z: 333.3 [M+H]⁺.

Methyl 2-amino-4-methyl-5-[(3,4,5-trimethoxyphenyl)carbamoyl]thiophene-3-carboxylate (Compound 18): ¹H NMR (400 MHz, d6-DMSO) δ 9.55 (s, 1H), 7.75 (brs, 2H), 7.04 (s, 2H), 3.75 (s, 3H), 3.73 (s, 6H), 3.62 (s, 3H), 3.32 (s, 3H); LC-MS (ESI) m/z: 381.3 [M+H]⁺.

Methyl 2-amino-4-methyl-5-(1,3-thiazol-2-ylcarbamoyl)thiophene-3-carboxylate (Compound 19): ¹H NMR (400 MHz, CDCl₃) δ 7.39 (d, 1H), 6.81 (d, 1H), 6.59 (brs, 2H), 3.87 (s, 3H), 2.72 (s, 3H); LC-MS (ESI) m/z: 298.2 [M+H]⁺.

Methyl 2-amino-5-[(3-methoxyphenyl)carbamoyl]-4-methylthiophene-3-carboxylate (Compound 20): ¹H NMR (300 MHz, CDCl₃) δ 7.34-7.33 (m, 2H), 7.23 (dd, 1H), 6.97 (dd, 1H), 6.68 (dd, 1H), 6.46 (brs, 2H), 3.86 (s, 3H), 3.82 (s, 3H), 2.66 (s, 3H); LC-MS (ESI) m/z: 321.2 [M+H]⁺.

Methyl 2-amino-5-[(2,4-dimethylphenyl)carbamoyl]thiophene-3-carboxylate (Compound 22): ¹H NMR (300 MHz, CDCl₃) δ 7.64 (d, 1H), 7.48 (s, 1H), 7.29-7.26 (m, 1H), 7.03 (s, 2H), 6.35 (s, 2H), 3.84 (s, 3H), 2.30 (s, 3H), 2.28 (s, 3H); LC-MS (ESI) m/z: 305.1 [M+H]⁺.

Ethyl 2-amino-5-[(2,4-dimethylphenyl)carbamoyl]-4-methylthiophene-3-carboxylate (Compound 25): ¹H NMR (400 MHz, CDCl₃) δ 7.73 (d, 1H), 7.13 (s, 1H), 7.04-7.02 (m, 2H), 6.42 (brs, 2H), 4.33 (q, 2H), 2.69 (s, 3H), 2.30 (s, 3H), 2.26 (s, 3H), 1.38 (t, 3H); LC-MS (ESI) m/z: 333.1 [M+H]⁺.

Ethyl 2-amino-5-{[3-(ethoxycarbonyl)phenyl]carbamoyl}-4-methylthiophene-3-carboxylate (Compound 26): ¹H NMR (300 MHz, CDCl₃) δ 8.00-7.93 (m, 2H), 7.81-7.78 (m, 1H), 7.44-7.39 (m, 2H), 6.50 (brs, 2H), 4.41-4.29 (m, 4H), 2.69 (s, 3H), 1.41-1.36 (m, 6H); LC-MS (ESI) m/z: 377.2 [M+H]⁺.

tert-Butyl 2-amino-5-[(2,4-dimethylphenyl)carbamoyl]-4-methylthiophene carboxylate (Compound 34): ¹H NMR (300 MHz, CDCl₃) δ 7.72 (d, 1H), 7.12 (brs, 1H), 7.04-7.02 (m, 2H), 6.39 (brs, 2H), 2.67 (s, 3H), 2.30 (s, 3H), 2.26 (s, 3H), 1.58 (s, 9H); LC-MS (ESI) m/z: 361.3 [M+H]⁺.

Methyl 2-amino-5-[(2-methoxyphenyl)carbamoyl]-4-methylthiophene-3-carboxylate (Compound 35): ¹H NMR (300 MHz, CDCl₃) δ 8.39 (dd, 1H), 8.17 (brs, 1H), 7.08-6.96 (m, 2H), 6.90 (dd, 1H), 6.43 (brs, 2H), 3.91 (s, 3H), 3.86 (s, 3H), 2.70 (s, 3H); LC-MS (ESI) m/z: 321.2 [M+H]⁺.

Preparation of methyl 2-amino-5-[(4-hydroxyphenyl)carbamoyl]-4-methylthiophene-3-carboxylate (Compound 21)
Step 1. Methyl 2-amino-5-[(4-{[tert-butyl(dimethyl)silyl]oxy}phenyl)carbamoyl]-4-methylthiophene-3-carboxylate

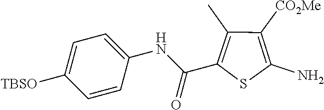 [SMILES: COC(=O)c1c(N)sc(C(=O)Nc2ccc(O[Si](C)(C)C(C)(C)C)cc2)c1C]

Methyl 2-amino-5-[(4-{[tert-butyl(dimethyl)silyl]oxy}phenyl)carbamoyl]-4-methylthiophene-3-carboxylate was prepared in a manner similar to that described above using N-(4-{[tert-butyl(dimethyl)silyl]oxy}phenyl)-3-oxobutanamide, methyl cyanoacetate, morpholine, and sulfur in anhydrous methanol (“MeOH”).

Step 2. Methyl 2-amino-5-[(4-hydroxyphenyl)carbamoyl]-4-methylthiophene-3-carboxylate

A 1 M solution of tetra-n-butylammonium fluoride in tetrahydrofuran (“THF”, 0.48 mL) was added to a solution of methyl 2-amino-5-[(4-{[tert-butyl(dimethyl)sily]oxy}phenyl)carbamoyl]-4-methylthiophene-3-carboxylate (100 mg, 0.24 mmol) in THF (1 mL) at 0° C. The reaction mixture was warmed to room temperature gradually and stirred for 3 h. The solvent was removed under reduced pressure. A saturated aqueous solution of NH₄Cl was added to the residue and the resulting mixture was extracted with CH₂Cl₂. The organic layer was dried over Na₂SO₄, filtered, and concentrated in vacuo. The residue was purified by column chromatography (EtOAc/CH₂Cl₂=⅓) to provide methyl 2-amino-5-[(4-hydroxyphenyl)carbamoyl]-4-methylthiophene-3-carboxylate (61 mg, 84%) as a white solid.

¹H NMR (400 MHz, CD₃OD) δ 7.32 (d, 2H), 6.75 (d, 2H), 3.82 (s, 3H), 2.55 (s, 3H). LC-MS (ESI) m/z: 307.1 [M+H]⁺.

Preparation of methyl 2-amino-4-bromo-5-[(2,4-dimethylphenyl)carbamoyl]thiophene-3-carboxylate (Compound 23)
Step 1. Methyl 4-bromo-2-{[(9H-fluoren-9-ylmethoxy)carbonyl]amino}-5-formylthiophene-3-carboxylate

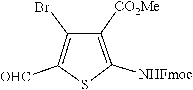

Anhydrous dimethylformamide (“DMF”, 60 μL, 0.76 mmol) was added to a solution of POBr₃ (470 mg, 1.64 mmol) in anhydrous CH₂Cl₂ (7.5 mL) at 0° C. and stirred for 1 h. A solution of methyl 2-{[9H-fluoren-9-ylmethoxy)carbonyl]amino}-4-oxo-4,5-dihydrothiophene-3-carboxylate (100 mg, 0.25 mmol) in anhydrous CH₂Cl₂ (7.5 mL) was added and the resulting mixture was then heated at 50° C. for 17 h. The reaction was cooled in an ice-bath and neutralized with a 2N aqueous solution of NaOH. The mixture was extracted with CH₂Cl₂. The organic layer was dried over MgSO₄, filtered, and concentrated in vacuo. The residue thus obtained was purified by column chromatography (EtOAc/Hexanes= 1/30 to 1/20) to provide methyl 4-bromo-2-{[(9H-fluoren-9-ylmethoxy)carbonyl]amino}-5-formylthiophene-3-carboxylate (77 mg, 62%). Step 2. 3-Bromo-5-{[(9H-fluoren-9-ylmethoxy)carbonyl]amino}-4-(methoxycarbonyl)-thiophene-2-carboxylic acid

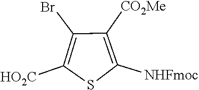

A reaction mixture of methyl 4-bromo-2-{[(9H-fluoren-9-ylmethoxy)carbonyl]-amino}-5-formylthiophene-3-carboxylate (77 mg, 0.16 mmol), 2-methyl-2-butene (0.2 mL, 1.58 mmol), sodium phosphate (56 mg, 0.47 mmol), and sodium chlorite (43 mg, 0.47 mmol) in 1,4-dioxane/H₂O (3/0.2, 3.2 mL) was stirred at room temperature for 3 h. The mixture was then extracted with EtOAc. The organic layer was dried over MgSO₄, filtered, and concentrated in vacuo to provide 3-bromo-5-{[(9H-fluoren ylmethoxy)carbonyl]amino}-4-(methoxycarbonyl)thiophene-2-carboxylic acid (75 mg, 95%).

Step 3. Methyl 4-bromo-5-[(2,4-dimethylphenyl)carbamoyl]-2-{[(9H-fluoren-9-ylmethoxy)carbonyl]amino}thiophene-3-carboxylate

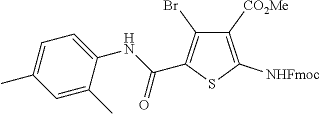

Four drops of anhydrous DMF were added to a solution of 3-bromo-5-{[(9H-fluoren-9-ylmethoxy)carbonyl]amino}-4-(methoxycarbonyl)thiophene-2-carboxylic acid (417 mg, 0.83 mmol) and oxalyl chloride (0.25 mL, 2.9 mmol) in CH₂Cl₂ (20 mL) at 0° C. The reaction mixture was warmed to room temperature gradually and stirred for 4 h, followed by concentration in vacuo to provide the acyl chloride intermediate. K₂CO₃ (137 mg, 1 mmol) and 2,4-dimethylaniline (92 μL, 0.74 mmol) were sequentially added to a solution of the acyl chloride (258 mg, 0.5 mmol) in CH₂Cl₂ (10 mL) at 0° C. The reaction mixture was warmed to room temperature gradually and stirred for 20 h. Water was added. The resulting mixture was extracted with CH₂Cl₂. The organic layer was dried over MgSO₄, filtered, and concentrated in vacuo. The residue thus obtained was purified by column chromatography (CH₂Cl₂/Hexanes=1/1 to MeOH/CH₂Cl₂= 1/20 to 1/10) to provide methyl 4-bromo-5-[(2,4-dimethylphenyl)carbamoyl]-2-{[(9H-fluoren-9-ylmethoxy)carbonyl]-amino}thiophene-3-carboxylate (186 mg, 62%).

Step 4. Methyl 2-amino-4-bromo-5-[(2,4-dimethylphenyl)carbamoyl]thiophene-3-carboxylate

A mixture of methyl 4-bromo-5-[(2,4-dimethylphenyl)carbamoyl]-2-{[(9H-fluoren-9-ylmethoxy)carbonyl]amino}thiophene-3-carboxylate (90 mg, 0.15 mmol) and piperidine (20% in CH₂Cl₂, 1 mL) in CH₂Cl₂ (3 mL) was stirred at 0° C. for 1 h. A saturated NaHCO₃ aqueous solution was then added. The resulting mixture was extracted with CH₂Cl₂. The organic layer was dried over MgSO₄, filtered, and concentrated in vacuo. The residue thus obtained was purified by column chromatography (EtOAc/Hexanes/NH₄OH= 30/90/0.2) to provide methyl 2-amino bromo-5-[(2,4-dimethylphenyl)carbamoyl]thiophene-3-carboxylate (11 mg, 19%).

¹H NMR (400 MHz, CDCl₃) δ 8.88 (brs, 1H), 7.88 (d, 1H), 7.04-7.03 (m, 2H), 6.56 (brs, 2H), 3.90 (s, 3H), 2.34 (s, 3H), 2.30 (s, 3H). LC-MS (ESI) m/z: 405.0 [M+Na]⁺.

Preparation of methyl 2-amino-5-[(2,4-dimethylphenyl)acetyl]-4-methylthiophene-3-carboxylate (Compound 24)
Step 1. Methyl 5-[(2,4-dimethylphenyl)acetyl]-2-[(4-methoxybenzyl)amino]-4-methylthiophene-3-carboxylate

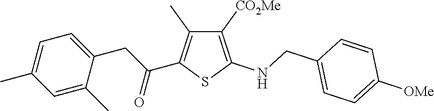 [SMILES: COC(=O)c1c(NCc2ccc(OC)cc2)sc(C(=O)Cc2ccc(C)cc2C)c1C]

Methyl acetoacetate (0.11 mL, 0.97 mmol) was added to a suspension of NaH (50 mg, 1.25 mmol) in DMF (4 mL) at 0° C. The resulting mixture was then warmed to room temperature and stirred for 30 min, followed by addition of 4-methoxybenzyl isothiocyanate (0.17 mL, 1.03 mmol). After stirred for 1.5 h, 1-bromo-3-(2,4-dimethyl-phenyl)propan-2-one (285 mg, 1.18 mmol) and K₂CO₃ (138 mg, 0.97 mmol) were sequentially added. The resulting mixture was stirred for 3 h and then quenched with water and extracted with EtOAc. The organic layer was dried over Na₂SO₄, filtered, and concentrated in vacuo. The residue thus obtained was purified by column chromatography (EtOAc/Hexanes= 1/12) to provide methyl 5-[(2,4-dimethylphenyl)-acetyl]-2-[(4-methoxybenzyl)amino]-4-methylthiophene-3-carboxylate (181 mg, 41%). Step 2. Methyl 2-amino-5-[(2,4-dimethylphenyl)acetyl]-4-methylthiophene-3-carboxylate A mixture of methyl 5-[(2,4-dimethylphenyl)acetyl]-2-[(4-methoxybenzyl)-amino]-4-methylthiophene-3-carboxylate (41 mg, 0.13 mmol) and triflic acid (“TfOH”, 115 μL, 1.3 mmol) in CH₂Cl₂ (1 mL) was stirred at 0° C. for 1 h. A saturated aqueous solution of NaHCO₃ was added to quench the reaction. The resulting mixture was extracted with CH₂Cl₂. The organic layer was dried over Na₂SO₄, filtered, and concentrated in vacuo. The residue thus obtained was purified by column chromatography (EtOAc/Hexanes= 1/10 to ⅕) to provide methyl 2-amino-5-[(2,4-dimethyl-phenyl)acetyl]-4-methylthiophene-3-carboxylate (21 mg, 51%).

¹H NMR (400 MHz, CDCl₃) δ 7.02-6.94 (m, 3H), 6.57 (brs, 2H), 3.94 (s, 2H), 3.85 (s, 3H), 2.71 (s, 3H), 2.29 (s, 3H), 2.23 (s, 3H). LC-MS (ESI) m/z: 318.1 [M+H]⁺.

Preparation of 2-amino-5-[(2,4-dimethylphenyl)carbamoyl]-4-methylthiophene-3-carboxylic acid (Compound 27)
Step 1: Benzyl 2-amino-5-[(2,4-dimethylphenyl)carbamoyl]-4-methylthiophene-3-carboxylate

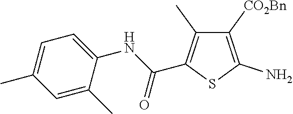 [SMILES: Cc1ccc(NC(=O)c2sc(N)c(C(=O)OCc3ccccc3)c2C)c(C)c1]

Benzyl 2-amino-5-[(2,4-dimethylphenyl)carbamoyl]-4-methylthiophene-3-carboxylate was prepared in a manner similar to that described above, using N-(2,4-dimethylphenyl)-3-oxobutyramide, benzyl cyanoacetate, morpholine, and sulphur in benzyl alcohol.

Step 2. 2-Amino-5-[(2,4-dimethylphenyl)carbamoyl]-4-methylthiophene-3-carboxylic acid

A mixture of benzyl 2-amino-5-[(2,4-dimethylphenyl)carbamoyl]-4-methylthio-phene-3-carboxylate (4 g, 10.1 mmol) and Pd(OH)₂ (0.71 g) in THF/EtOH ( 2/1, 75 mL) was stirred at room temperature under 1 atm H_{2(g)} for 12 h. The reaction mixture was filtered through a pad of Celite. The filtrate was concentrated under reduced pressure. The residue thus obtained was recrystallized with CH₂Cl₂ to provide 2-amino-5-[(2,4-dimethylphenyl)carbamoyl]-4-methylthiophene-3-carboxylic acid (2.3 g, 75%) as an off-white solid.

¹H NMR (400 MHz, d6-DMSO) δ 12.29 (brs, 1H), 8.99 (s, 1H), 7.72 (brs, 2H), 7.21 (d, 1H), 7.03 (s, 1H), 6.97 (d, 1H), 2.54 (s, 3H), 2.26 (s, 3H), 2.15 (s, 3H). LC-MS (ESI) m/z: 305.3 [M+H]⁺.

Preparation of 5-amino-N⁴-(cyanomethyl)-N²-(2,4-dimethylphenyl)-3-methylthiophene-2,4-dicarboxamide (Compound 28)
Step 1. Ethyl 2-[(tert-butoxycarbonyl)amino]-5-[(2,4-dimethylphenyl)carbamoyl]methylthiophene-3-carboxylate

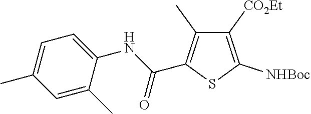 [SMILES: CCOC(=O)c1c(NC(=O)OC(C)(C)C)sc(C(=O)Nc2ccc(C)cc2C)c1C]

tert-Butyloxycarbonyl anhydride (“Boc₂O”, 0.23 mL, 1.02 mmol) was added slowly to a solution of Compound 25 (307 mg, 0.92 mmol), dimethylaminopyridine (“DMAP”, 11 mg, 0.09 mmol), and Et₃N (0.77 mL, 5.54 mmol) in THF (5 mL) at room temperature and stirred for 23 h. The reaction mixture was quenched with a saturated NaHCO₃ aqueous solution and extracted with EtOAc. The organic layer was dried over Na₂SO₄, filtered, and concentrated in vacuo. The residue thus obtained was purified by column chromatography (EtOAc/Hexanes=¼ to ½) to provide ethyl 2-[(tert-butoxycarbonyl)amino]-5-[(2,4-dimethylphenyl)carbamoyl]-4-methylthiophene-3-carboxylate (122 mg, 31%).

Step 2. 2-[(tert-Butoxycarbonyl)amino]-5-[(2,4-dimethylphenyl)carbamoyl]-4-methylthiophene-3-carboxylic acid

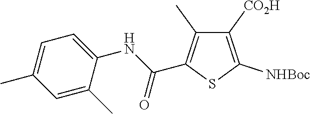 [SMILES: Cc1ccc(NC(=O)c2sc(NC(=O)OC(C)(C)C)c(C(=O)O)c2C)c(C)c1]

A solution of NaOH (85 mg, 2.12 mmol) in H₂O (2 mL) was added to a solution of ethyl 2-[(tert-butoxycarbonyl)amino]-5-[(2,4-dimethylphenyl)carbamoyl]-4-methyl-thiophene-3-carboxylate (458 mg, 1.06 mmol) in MeOH (2 mL) at room temperature. The resulting mixture was heated at 90° C. for 3 h and then cooled in an ice-bath. The reaction was acidified with a 2 N HCl aqueous solution. The resulting precipitate was filtered, collected, and dried in vacuo to provide 2-[(tert-butoxycarbonyl)-amino]-5-[(2,4-dimethylphenyl)carbamoyl]-4-methylthiophene-3-carboxylic acid (348 mg, 81%).

Step 3. tert-Butyl {3-[(cyanomethyl)carbamoyl]-5-[(2,4-dimethylphenyl)-carbamoyl]-4-methylthiophen-2-yl}carbamate

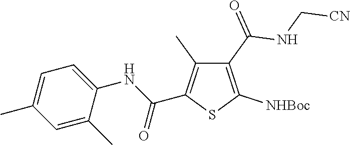 [SMILES: Cc1ccc(NC(=O)c2sc(NC(=O)OC(C)(C)C)c(C(=O)NCC#N)c2C)c(C)c1]

Et₃N (0.08 mL, 0.54 mmol) and a≥50 wt. % solution of propylphosphonic anhydride (“T₃P”) in EtOAc (0.29 mL, 0.27 mmol) was sequentially added to a solution of 2-[(tert-butoxycarbonyl)amino]-5-[(2,4-dimethylphenyl)carbamoyl]-4-methylthiophene-3-carboxylic acid (55 mg, 0.14 mmol) and aminoacetonitrile hydrochloride (19 mg, 0.2 mmol) in DMF (1.5 mL) at room temperature and stirred for 18 h, followed by quenched with water and extracted with EtOAc. The organic layer was dried over Na₂SO₄, filtered, and concentrated in vacuo. The residue was purified by column chromatography (EtOAc/Hexanes=3/1 to MeOH/CH₂Cl₂=1/30) to provide tert-butyl {3-[(cyanomethyl)carbamoyl]-5-[(2,4-dimethylphenyl)carbamoyl]-4-methylthiophen-2-yl}carbamate (17 mg, 28%).

Step 4. 5-Amino-N⁴-(cyanomethyl)-N²-(2,4-dimethylphenyl)-3-methylthiophene-2,4-dicarboxamide

A mixture of tert-butyl {3-[(cyanomethyl)carbamoyl]-5-[(2,4-dimethylphenyl)-carbamoyl]-4-methylthiophen-2-yl}carbamate (16 mg, 0.04 mmol) and ZnBr₂ (42 mg, 0.19 mmol) in CH₂Cl₂ (2 mL) was stirred at room temperature for 20 h. Water was added and the resulting mixture was extracted with CH₂Cl₂. The organic layer was dried over Na₂SO₄, filtered, and concentrated in vacuo. The residue was purified by column chromatography (EtOAc/Hexanes=1/1) to provide 5-amino-N⁴-(cyanomethyl)-N²-(2,4-dimethylphenyl)-3-methylthiophene-2,4-dicarboxamide (5 mg, 40%).

¹H NMR (300 MHz, CD₃OD) δ 7.18 (d, 1H), 7.08 (s, 1H), 7.02 (d, 1H), 4.27 (s, 2H), 2.57 (s, 3H), 2.30 (s, 3H), 2.23 (s, 3H). LC-MS (ESI) m/z: 343.1 [M+H]⁺.

Preparation of 5-amino-N-(2,4-dimethylphenyl)-4-(hydroxyacetyl)-3-methylthiophene-2-carboxamide (Compound 29)
Step 1. tert-Butyl {5-[(2,4-dimethylphenyl)carbamoyl]-3-[methoxy(methyl)carbamoyl]-4-methylthiophen-2-yl}carbamate

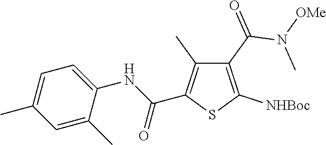 [SMILES: CON(C)C(=O)c1c(NC(=O)OC(C)(C)C)sc(C(=O)Nc2ccc(C)cc2C)c1C]

A mixture of 2-[(tert-butoxycarbonyl)amino]-5-[(2,4-dimethylphenyl)carbamoyl]-4-methylthiophene-3-carboxylic acid (542 mg, 1.34 mmol), N,O-dimethylhydroxylamine hydrochloride (196 mg, 2 mmol), 1-ethyl-3-(3-dimethylaminopropyl)carbodiimide (“EDC”, 308 mg, 1.61 mmol), hydroxybenzotriazole (“HOBt”, 181 mg, 1.34 mmol), and N,N-diisopropylethylamine (“DIPEA”, 0.8 mL, 4.69 mmol) in CH₂Cl₂ (20 mL) was stirred at room temperature for 21 h. Water was added and the resulting mixture was extracted with EtOAc. The organic layer was dried over MgSO₄, filtered, and concentrated in vacuo. The residue was purified by column chromatography (EtOAc/Hexanes=1/3 to 1/1) to provide tert-butyl {5-[(2,4-dimethylphenyl)carbamoyl]-3-[methoxy(methyl)carbamoyl]-4-methylthiophen-2-yl}carbamate (268 mg, 45%).

Step 2. tert-Butyl {3-[(benzyloxy)acetyl]-5-[(2,4-dimethylphenyl)carbamoyl]-4-methylthiophen-2-yl}carbamate

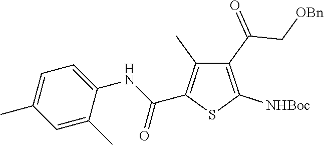 [SMILES: Cc1ccc(NC(=O)c2sc(NC(=O)OC(C)(C)C)c(C(=O)COCc3ccccc3)c2C)c(C)c1]

A solution of tert-butyl {5-[(2,4-dimethylphenyl)carbamoyl]-3-[methoxy(methyl)-carbamoyl]-4-methylthiophen-2-yl}carbamate (197 mg, 0.44 mmol) in anhydrous THF (7 mL) was added slowly to an in situ prepared solution of benzyloxymethyl magnesium chloride in THF (1.44 M, 5 mL) at −60° C. The reaction was warmed to room temperature gradually and stirred for 21 h, followed by quenching with a saturated aqueous solution of NH₄Cl and extraction with EtOAc. The organic layer was dried over MgSO₄, filtered, and concentrated in vacuo. The residue thus obtained was purified by column chromatography (EtOAc/CH₂Cl₂= 1/100 to 1/20) to provide tert-butyl {3-[(benzyloxy)acetyl]-5-[(2,4-dimethylphenyl)carbamoyl]-4-methylthiophen-2-yl}carbamate (173 mg, 77%).

Step 3. tert-Butyl {5-[(2,4-dimethylphenyl)carbamoyl]-3-(hydroxyacetyl)-4-methylthiophen-2-yl}carbamate

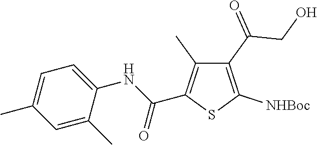 [SMILES: Cc1ccc(NC(=O)c2sc(NC(=O)OC(C)(C)C)c(C(=O)CO)c2C)c(C)c1]

tert-Butyl {3-[(benzyloxy)acetyl]-5-[(2,4-dimethylphenyl)carbamoyl]methylthiophen-2-yl}carbamate (103 mg, 0.2 mmol) was hydrogenated over 10% Pd/C (80 mg) in a mixed solvent (EtOH/EtOAc=2/3, 5 mL) at room temperature for 17 h. The reaction mixture was filtrated through Celite. The filtrate was concentrated in vacuo. The residue thus obtained was purified by column chromatography (EtOAc/Hexanes=⅓ to ½) to provide tert-butyl {5-[(2,4-dimethylphenyl)carbamoyl]-3-(hydroxyacetyl)-4-methylthiophen-2-yl}carbamate (43 mg, 51%).

Step 4. 5-Amino-N-(2,4-dimethylphenyl)-4-(hydroxyacetyl)-3-methylthiophene-2-carboxamide

A 50% solution of TFA in CH₂Cl₂ (1 mL) was added to a solution of tert-butyl {5-[(2,4-dimethylphenyl)carbamoyl]-3-(hydroxyacetyl)-4-methylthiophen-2-yl}carbamate (43 mg, 0.1 mmol) in CH₂Cl₂ (2 mL) at 0° C. The reaction mixture was stirred at room temperature for 3 h, quenched with a saturated aqueous solution of NaHCO₃, and then extracted with CH₂Cl₂. The organic layer was dried over MgSO₄, filtered, and concentrated in vacuo. The residue was recrystallized from CH₂Cl₂/Hexanes to provide 5-amino-N-(2,4-dimethylphenyl)-4-(hydroxyacetyl)-3-methylthiophene-2-carboxamide (9.0 mg, 27%).

¹H NMR (400 MHz, d6-DMSO) δ 9.18 (s, 1H), 8.50 (s, 2H), 7.16 (d, 1H), 7.02 (s, 1H), 6.96 (d, 1H), 4.71 (t, 1H), 4.47 (d, 2H), 2.53 (s, 3H), 2.24 (s, 3H), 2.13 (s, 3H). LC-MS (ESI) m/z: 319.1 [M+H]⁺.

Preparation of 4-acetyl-5-amino-N-(2,4-dimethylphenyl)-3-methylthiophene-2-carboxamide (Compound 30) Step 1. Benzyl {[2-acetyl-1-(benzylamino)-3-oxobut-1-en-1-yl]sulfanyl}acetate

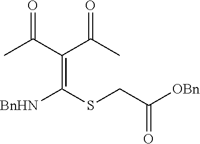

NaH (419 mg, 10.5 mmol) was added to an acetylacetone (1 g, 10 mmol) solution in DMF (10 mL) at 0° C. After 30 min, benzyl isothiocyanate (1.32 mL, 10 mmol) was added. The resulting reaction mixture was warmed to room temperature gradually and stirred for 3 h. Benzyl bromoacetate (1.72 mL, 11 mmol) was added and stirred overnight, followed by quenching with water and extraction with EtOAc. The organic layer was dried over MgSO₄, filtered, and concentrated in vacuo. The residue thus obtained was purified by column chromatography (EtOAc/Hexanes=1/20) to provide benzyl {[2-acetyl-1-(benzylamino)-3-oxobut-1-en-1-yl]sulfanyl}acetate (2.18 g, 55%). Step 2. Benzyl 4-acetyl-5-(benzylamino)-3-methylthiophene-2-carboxylate

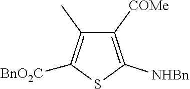

A solution of benzyl {[2-acetyl-1-(benzylamino)-3-oxobut-1-en-1-yl]sulfanyl}-acetate (2.13 g, 5.36 mmol) and K₂CO₃ (1.1 g, 8.03 mmol) in THF (40 mL) was heated at 75° C. for 3 h. The reaction mixture was cooled to room temperature, quenched with water, and extracted with EtOAc. The organic layer was dried over MgSO₄, filtered, and concentrated in vacuo. The residue thus obtained was purified by column chromatography (EtOAc/Hexanes= 1/20 to 1/10) to provide benzyl 4-acetyl-5-(benzylamino)-3-methylthiophene-2-carboxylate (1.44 g, 71%).

Step 3. 4-Acetyl-5-(benzylamino)-3-methylthiophene-2-carboxylic acid

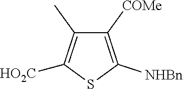

A mixture of benzyl 4-acetyl-5-(benzylamino)-3-methylthiophene-2-carboxylate (682 mg, 1.8 mmol) and a 7 N aqueous solution of KOH (4 mL) in THF (20 mL) was heated at 100° C. for 24 h. The reaction mixture was cooled to 0° C., acidified with a 2 N HCl aqueous solution, and extracted with EtOAc. The organic layer was dried over MgSO₄, filtered, and concentrated in vacuo. The residue thus obtained was purified by column chromatography (MeOH/CH₂Cl₂= 1/50 to 1/20) to provide 4-acetyl-5-(benzylamino)-3-methylthiophene-2-carboxylic acid (315 mg, 61%).

Step 4. 4-Acetyl-5-(benzylamino)-N-(2,4-dimethylphenyl)-3-methylthiophene carboxamide

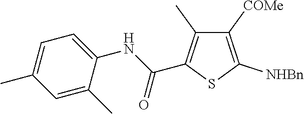

A mixture of 4-acetyl-5-(benzylamino)-3-methylthiophene-2-carboxylic acid (245 mg, 0.85 mmol), EDC (177 mg, 0.92 mmol), HOBt (104 mg, 0.77 mmol), 2,4-dimethylaniline (95 μL, 0.77 mmol), and DIPEA (0.46 mL, 2.69 mmol) in DMF (5 mL) was heated at 50° C. for 16 h, cooled to room temperature, quenched with water, and extracted with EtOAc. The organic layer was dried over MgSO₄, filtered, and concentrated in vacuo. The residue thus obtained was purified by column chromatography (EtOAc/CH₂Cl₂=1/100) to provide 4-acetyl-5-(benzylamino)-N-(2,4-dimethylphenyl)-3-methylthiophene-2-carboxamide (140 mg, 42%).

Step 5. 4-Acetyl-5-amino-N-(2,4-dimethylphenyl)-3-methylthiophene-2-carboxamide

A mixture of 4-acetyl-5-(benzylamino)-3-methyl-N-phenylthiophene-2-carboxamide (60 mg, 0.15 mmol) and trifluoromethanesulfonic acid (0.13 mL, 1.53 mmol) in CH₂Cl₂ (3 mL) was heated at 60° C. for 1 h, cooled in an ice-bath, neutralized with a 6 N NaOH aqueous solution, and then extracted with EtOAc. The organic layer was dried over MgSO₄, filtered, and concentrated in vacuo. The residue was purified by column chromatography (EtOAc/CH₂Cl₂=¼) to provide 4-acetyl-5-amino-N-(2,4-dimethyl-phenyl)-3-methylthiophene-2-carboxamide (8.4 mg, 18%).

¹H NMR (300 MHz, d6-DMSO) δ 9.16 (s, 1H), 8.47 (s, 2H), 7.19 (d, 1H), 7.04 (s, 1H), 6.98 (d, 1H), 2.59 (s, 3H), 2.42 (s, 3H), 2.26 (s, 3H), 2.15 (s, 3H). LC-MS (ESI) m/z: 303.1 [M+H]⁺.

### Compounds 31 and 32

Both compounds were prepared in a manner similar to that for Compound 30.

4-Acetyl-5-amino-3-methyl-N-phenylthiophene-2-carboxamide (Compound 31): ¹H NMR (400 MHz, d6-DMSO) δ 9.80 (s, 1H), 8.50 (s, 2H), 7.60 (d, 2H), 7.31 (dd, 2H), 7.06 (dd, 1H), 2.54 (s, 3H), 2.42 (s, 3H); LC-MS (ESI) m/z: 275.1 [M+H]⁺.

4-Acetyl-5-amino-N-(4-fluorophenyl)-3-methylthiophene-2-carboxamide (Compound 32): ¹H NMR (400 MHz, CD₃OD) δ 7.57-7.53 (m, 2H), 7.09-7.04 (m, 2H), 2.62 (s, 3H), 2.49 (s, 3H); LC-MS (ESI) m/z: 293.0 [M+H]⁺.

### Compound 33

A mixture of Compound 1 (100 mg, 0.31 mmol) and acetic anhydride (“Ac₂O”; 10 mL) was stirred under a reflux condition for 2 h. The reaction was then concentrated under reduced pressure and the resulting crude was recrystallized with hot MeOH to afford Compound 33 as an off-white solid.

¹H NMR (400 MHz, d6-DMSO) δ 11.04 (s, 1H), 9.53 (s, 1H), 7.18 (d, 1H), 7.06 (s, 1H), 6.99 (d, 1H), 3.87 (s, 3H), 2.55 (s, 3H), 2.27 (s, 3H), 2.26 (s, 3H), 2.17 (s, 3H); LC-MS (ESI) m/z: 361.2 [M+H]⁺.

### EXAMPLE 2: Cisd2-Luciferase Reporter Assay

Compounds 1-35 were tested for increasing the level of Cisd2 in human embryonic kidney cells 293 (HEK293), a transfected cell line expressing Cisd2, using the Cisd2-luciferase reporter assay as described below.

The transfected HEK293 cells stably expressed Cisd2 promoter with firefly luciferase reporter (“HEK293-CISD2”). They were kept in Dulbecco's modified Eagle's medium (“DMEM”) (Gibco, New York, N.Y.). Culture medium was supplemented with 10% heat-inactivated fetal bovine serum (“FBS”), 2 mM L-Glutamine, 1 mM sodium pyruvate, 0.1% MEM Non-Essential Amino Acids, 1 μg/ml puromycin and 1% penicillin/streptomycin (HyClone, Logan, Utah). The cells were maintained at 37° C. in an incubator (Thermo Fisher Scientific, Waltham, Mass.) with an atmosphere of 5% CO₂. The stably transfected cells were seeded in 384-well plates at a density of 3×10³ cells/well. After incubation with a tested compound at 37° C. for 24 h, ONE-Glo™ Luciferase Assay System (Promega, Madison, Wis.) was added at room temperature for 10 minutes to detect any luciferase activity. Luminescence intensity was registered using plate reader BioTek Synergy Neo2™ (Agilent, Santa Clara, Calif.).

Each of Compounds 1-35 was found to activate Cisd2 to a degree indicated by its EC₅₀ value. See Table 2 below. EC₅₀ values in this table are presented in three classes, i.e., A: <1 μM and B: 1 μM-10 μM.

TABLE 2

| Compound No. | EC₅₀ |
|---|---|
| 1 | A |
| 2 | B |
| 3 | A |
| 4 | B |
| 5 | A |
| 6 | A |
| 7 | A |
| 8 | A |
| 9 | A |
| 10 | A |
| 11 | A |
| 12 | A |
| 13 | A |
| 14 | B |
| 15 | A |
| 16 | A |
| 17 | B |
| 18 | B |
| 19 | A |
| 20 | A |
| 21 | A |
| 22 | B |
| 23 | A |
| 24 | B |
| 25 | A |
| 26 | B |
| 27 | A |
| 28 | B |
| 29 | A |
| 30 | A |
| 31 | A |
| 32 | A |
| 33 | B |
| 34 | B |
| 35 | B |

### EXAMPLE 3: In Vivo Study for Treating Anti-Nonalcoholic Fatty Liver Disease

Compound 1 was evaluated for its anti-nonalcoholic fatty liver disease (“NAFLD”) effect and in vivo toxicity following an assay described below.

Cisd2 floxed allele (Cisd2 f/f) mice were prepared following a procedure described in Wang et al., Hum. Mol. Genet.23, 4770-85 (2014). To obtain Cisd2 heterozygous hepatocyte-specific knockout (Cisd2 hKO-het, Cisd2 f/+; Alb-Cre Tg), Cisd2 f/f mice were bred with Albumin-Cre transgenic (Alb-Cre Tg, JAX003574) mice. After one generation, Cisd2 hKO-het mice were obtained. Male mice were chosen to be used in this study. All mice have a pure or congenic C₅₇BL/6 background and were bred/housed in a specific pathogen-free facility with a 12 h light/12 h dark cycle at a temperature of 20-22° C. Groups of mice at 8 weeks old were provided a diet (AIN93G, TestDiet) containing a vehicle (0.14 wt % of DMF and 0.15 wt % of Cremophor® EL) or compound 1 (0.015 wt %) for 4 weeks.

Histopathology was used to evaluate the efficacy of Compound 1 in ameliorating NAFLD. Liver tissues were collected and fixed with 10% neutral buffered formalin at 4° C. overnight. They were then processed in a tissue processor (STP120, Microm, Thermo Fisher Scientific Inc., Waltham, Mass.), followed by embedding in paraffin. H&E of tissue sections (3-4 μm) were carried out by a standard protocol. Specifically, the liver tissue sections were deparaffinized, rehydrated, and stained by Mayer's hematoxylin (Muto Pure Chemicals Co., Bunkyo-ku, Japan) and 1% eosin Y solution (Muto Pure Chemicals Co.). The stained liver tissue sections were then dehydrated, mounted with a mounting medium (Surgipath), and coverslipped.

Serum biochemical analysis was performed to evaluate in vivo toxicity of Compound 1. Whole blood samples were collected from facial vein or cardiac puncture at sacrifice for analyzing serum alanine aminotransferase (GPT/ALT), aspartate aminotransferase (GOT/AST), blood urea nitrogen (BUN) and creatinine (CRE) levels using Fuji Dri-Chem 3500s analyzer (Fujifilm Corp., Tokyo, Japan).

The results showed that Compound 1 significantly enhanced the expression levels of Cisd2 protein in the Cisd2+/− mice (namely, a protein expression level of 0.7 in mice treated with Compound 1 as compared to a protein level of only 0.43 in the control group) and ameliorated NAFLD and liver pathology (steatosis, hepatocyte disorganization and ballooning) caused by Cisd2 haploinsufficiency without detectable liver and kidney toxicity.

### EXAMPLE 4: In Vivo Study of Treating Nonalcoholic Steatohepatitis

Compound 1 was used to alleviate nonalcoholic steatohepatitis (NASH) in wild-type and Cisd2 hKO male mice described above.

Cisd2 hKO were bred for two generations. All male mice used in this study had a pure or congenic C₅₇BL/6 background and were bred/housed in a specific pathogen-free facility with a 12 h light/12 h dark cycle at a temperature of 20-22° C. They were fed 30% (w/v) fructose (Alfa Aesar, Tewksbury, Mass.) in drinking water to establish a fructose-induced NASH model. Groups of mice at 2 months old were provided with a diet (AIN93G, TestDiet, St. Louis, Mo.) containing a vehicle (0.14 wt % of DMF and 0.15 wt % of Cremophor® EL) or Compound 1 (0.015 wt %) for 5 months. The food and drinking water were provided ad libitum.

Histopathology was used to evaluate the efficacy of Compound 1 in alleviating NASH. Liver tissues were processed and stained as described in Example 3 above.

An oral glucose tolerance test (GTT) and an insulin tolerance test (ITT) were performed to study beneficial effects of Compound 1 in the fructose-induced glucose intolerance and hepatic insulin resistant mouse model. In the oral glucose tolerance test, mice were fasted for 14 hours (7 p.m. to 9 a.m.) and then were orally administrated with glucose water (1.5 mg/g body weight) using a feeding needle. Blood samples were collected from tail vein before (0 min) and after glucose treatment at predetermined time points. Blood glucose levels were measured using OneTouch® Ultra glucose test strips and SureStep® Meter (LifeScan, Milpitas, Calif.). In the insulin tolerance test, mice were fasted for 2 h (9 a.m. to 11 a.m.) and then intraperitoneally injected with insulin (0.75 U/kg body weight) (Actrapid® human regular insulin, Novo Nordisk, Bagsverd, Denmark). Blood samples were collected and monitored at predetermined time points. The results showed that Compound 1 significantly increased Cisd2 protein levels (a protein level of 0.8 in mice treated with Compound 1 vs. 0.4 in the control group), ameliorated fructose-induced NASH, and alleviated fructose-induced glucose intolerance and insulin resistance in WT mice without obvious toxicity. On the other hand, Compound 1 has no overt beneficial effect in the hepatocyte-specific Cisd2 knockout (Cisd2 hKO) mice. The result suggests that the effect of Compound 1 is mainly dependent on a molecular mechanism/pathway involving Cisd2.

### EXAMPLE 5: In Vivo Study of Protecting Against Doxorubicin-Induced Cardiotoxicity

Compound 1 was evaluated for protecting against doxorubicin-induced cardiotoxicity in mice.

Male C₅₇BL/6 mice between 7 and 8 months old were treated with Compound 1 (at a dosage of 1 mg/kg) or a vehicle (3.3% DMSO in normal saline) via intraperitoneal injection (i.p.) twice a day for 2 days. At day 0, 1 hour after the treatment with Compound 1, doxorubicin (25 mg/kg, i.p.) or another vehicle (8% DMSO in normal saline) was injected into mice.

Electrocardiogram (ECG) and heart echo on day 4 and day 5 respectively were used as indications of cardiac function. Mice tissues were collected on day 5 and were fixed with 10% neutral buffered formalin at 4° C. overnight, followed by paraffin embedding and sectioning.

Functional test of heart using ECG was performed as described in Yeh et al., PLoS Biol. 2019, 17, e3000508. The mice were maintained on a 12:12 hour dark-light cycle with lights switched on at 6:00 am. All procedures took place during the light phase. Anesthesia was initially induced by placing the mice for 3-5 minutes in a chamber filled with 3 v/v % isoflurane (Aesica Pharmaceuticals, Hertfordshire, UK). The mice were then positioned on a warm pad (ALA Scientific Instruments Inc., East Farmingdale, N.Y.) that enabled maintaining temperature during ECG recording. Mice breathed freely through a nose cone. Anesthesia was maintained by inhalation of 1.5% isoflurane. Continuous 5-minute ECGs were obtained using subcutaneous electrodes attached at the four limbs and recorded with PowerLab® data acquisition system (model ML866, ADInstruments, Colorado Springs, Colo.) and Animal Bio Amp® (model ML136, ADInstruments). ECG analysis was performed in an unbiased fashion where 1500 beats were analyzed using LabChart® 7 Pro version 7.3.1 (ADInstruments). Detection and analysis of QTc interval, QRS intervals, Tpeak-T end intervals were set to Mouse ECG parameters. The values thus obtained were compared statistically by utilizing the Mann-Whitney U test, and p<0.05 was accepted as significant.

Transthoracic mouse echocardiography was used to provide noninvasive imaging of the heart on a VisualSonics® VeVo® 2100 imaging system (VisualSonics, Toronto, Ontario, Canada). Male mice were anesthetized with 1% isoflurane in 95% O_{2(g)}. Body temperature was maintained and monitored at 36° C. to 37° C. on a heated pad (TC-1000, CWE Inc. USA). ECGs were continuously monitored. Cardiac function was assessed using a high-frequency 30-50 MHz probe, as described in Casaclang-Verzosa et al., J. Vis. Exp. 120, e54110 (2017). Data analysis was performed using VisualSonics® software. Personnel responsible for data acquisition were blinded to the animal grouping.

The results showed that Compound 1 significantly increased Cisd2 protein levels, rescued the abnormality of ECG induced by doxorubicin, and attenuated the doxorubicin-induced abnormal cardiac conductance in ST height and QRS interval. In addition, the global cardiac performance test showed that Compound 1 effectively maintained the cardiac performance as compared to damages caused by a doxorubicin injection. The results indicate that Compound 1 unexpectedly protected against doxorubicin-induced cardiotoxicity.

### EXAMPLE 6: In Vivo Study of Treating Doxorubicin-Induced Cachexia and Hepatotoxicity

Compound 1 was used in rescuing doxorubicin-induced cachexia and hepatotoxicity in the following in vivo study.

Male C₅₇BL/6 mice between 7 and 8 months were pretreated with Compound 1 (1 mg/kg) or a vehicle (3.3% DMSO in normal saline) via intraperitoneal injection (i.p.) twice a day for 2 days. At day 0, 1 hour after the treatment with Compound 1, doxorubicin (25 mg/kg, i.p.) or another vehicle (8% DMSO in normal saline) were injected into mice.

Different tissues including livers, cardiac muscle (left ventricle), skeletal muscle (gastrocnemius), brown adipose tissue, and white adipose tissue were weighted and collected after mice were sacrificed. The tissues were fixed with 10% neutral buffered formalin at 4° C. overnight, processed in a tissue processor, and then embedded in paraffin. H&E of tissue sections (3-4 μm) were carried out by a standard protocol as described above.

The results showed that Compound 1 alleviated doxorubicin-induced cardiac damage at the histopathological level, including cytosolic vacuolation of cardiomyocytes and degeneration of muscle fibers. Additionally, the global cardiac weight loss after a single high-dose of doxorubicin injection was attenuated by Compound 1. Unexpectedly, Compound 1 alleviated doxorubicin-induced hepatotoxicity and cachexia.

### EXAMPLE 7: In Vivo Study of Treating Age-Related Arrhythmogenesis

Compound 1 was used in rescuing age-related arrhythmogenesis in following in vivo study.

Female C₅₇BL/6 mice between 20 and 21 months old were pretreated with Compound 1 (1 mg/kg) or a vehicle (3.3% DMSO in normal saline) via intraperitoneal injection (i.p.) twice a day for 18 days.

Electrocardiogram (ECG) and heart echo were examined 2-3 days before and after 18-day compound treatment as indications of cardiac function. Mice tissues were collected on day 5 and were fixed with 10% neutral buffered formalin at 4° C. overnight, followed by paraffin embedding and sectioning.

Functional test of heart using ECG and Transthoracic mouse echocardiography were processed as described in Example 5 above.

The study on aging-related cardiac dysfunction in post-menopausal aging mice revealed that the widened QTc, prolonged Tpeak-Tend, and irregular PR interval could be preserved with short-term Compound 1 injection. The systolic contractility remained the same during aging with Compound 1 injection. The systolic (measuring ejection fraction) and diastolic (measuring Myocardial performance index) function of heart was deteriorated with vehicle injection. The results showed unexpectedly that compound 1 protected against age-related arrhythmogenesis.

### EXAMPLE 8: In Vivo Study of Treating Age-Related Cardiac Pathological Change

Compound 1 was used in rescuing age-related arrhythmogenesis in following in vivo study.

Female C₅₇BL/6 mice between 20 and 21 months were pretreated with Compound 1 (1 mg/kg) or a vehicle (3.3% DMSO in normal saline) via intraperitoneal injection (i.p.) twice a day for 2 days. Electrocardiogram (ECG) and heart echo were examined 2-3 days before and after 18-day compound treatment as indications of cardiac function. Mouse tissues were collected on day 5 and were fixed with 10% neutral buffered formalin at 4° C. overnight, followed by paraffin embedding and sectioning.

Cardiac tissues were weighted and collected after mice were sacrificed. The tissues were fixed with 10% neutral buffered formalin at 4° C. overnight, processed in a tissue processor, and then embedded in paraffin. H&E and Sirius red stain of tissue sections (3-4 μm) were carried out by a standard protocol as described above.

The study on age-related cardiac pathological change revealed that the cytosolic vacuole appeared at aging was disappeared after Compound 1 treatment. Additionally, the endomysial fibrosis increase during ageing could be prevented with Compound 1. Unexpectedly, Compound 1 alleviated age-related cardiac pathological changes.

In addition to the aforementioned studies, increasing Cisd2 expression has been found to attenuate Aβ-mediated neuron loss in Alzheimer's disease, ameliorate muscle degeneration, and slow skin aging. See Chen et al., Journal of Pathology 250, 299-311 (2020); and Wu et al., Human Molecular Genetics 21(18), 3956-68 (2012). Further, an enhanced level of Cisd2 can also improve glucose intolerance. Moreover, Cisd2 upregulation can attenuate injury-induced inflammation in aged animals and inflammatory cell model. See Lin et al., Injury 46(12), 2341-50 (2015); and Lin et al., Nutrients 11(3), 700 (2019). As such, the thiophene compounds of formula (I) are also suitable for treating Alzheimer's disease, muscle degeneration, skin aging, glucose intolerance, and inflammation.

### EXAMPLE 9: In Vivo Study of Treating Hypertensive Cardiomyopathy

Compound 1 was used to ameliorate hypertensive cardiomyopathy in wild type female mice.

All female mice used in this study had a pure or congenic C₅₇BL/6 background and were bred/housed in a specific pathogen-free facility with a 12 h light/12 h dark cycle at a temperature of 20-22° C. Female C₅₇BL/6 mice between 11 and 12 months old received bilateral ovariectomy half a month before treatment. Groups of ovariectomized mice at 12 months old were provided with a diet (AIN93G, TestDiet) containing a vehicle (0.14 wt % of DMF and 0.15 wt % of Cremophor® EL) or Compound 1 (0.015 wt %) for 3 months. One month after treatment, one osmotic pump per month with angiotensin II (1 μg/kg/min) was implanted into all experimental animals for consecutive 2 months to establish a hypertensive cardiomyopathy model. The food and drinking water were provided ad libitum.

Systolic blood pressure (SBP), electrocardiogram (ECG), and heart echo before ovariectomy and 3 months after treatment respectively were used as indications of cardiovascular function. SBP was measured with tail cuff systems in conscious mice. ECG and cardiac echo were performed as described in Example 5 above. Mice tissues were collected after 3 months of treatment and were fixed with 10% neutral buffered formalin at 4° C. overnight, followed by paraffin embedding and sectioning. H&E and Sirius red stain of tissue sections (3-4 μm) were carried out by a standard protocol as described in Example 8 above.

The study on hypertension-induced cardiac dysfunction in post-menopausal mice revealed that Compound 1 significantly decreased SBP. The study also showed that the high P amplitude and bundle branch block could be preserved by feeding mice with Compound 1. In addition, the global cardiac performance test showed that Compound 1 effectively maintained cardiac performance as compared to damages caused by hypertension. The systolic contractility remained the same during hypertension with Compound 1 feeding. By contrast, the systolic function of the heart deteriorated with vehicle injection.

Further, it was observed that Compound 1 ameliorated hypertension-induced cardiac damage at the histopathological level, including hypertrophic change of ventricular wall and perivascular fibrosis. Unexpectedly, Compound 1 ameliorated hypertensive cardiomyopathy.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their efficacy in treating a Cisd2 insufficiency-associated disorder and increasing Cisd2 gene expression. Thus, other embodiments are also within the claims.

What is claimed is:

1. A method of treating a Cisd2 insufficiency-associated disorder, the method comprising:
identifying a subject suffering from a Cisd2 insufficiency-associated disorder, and
administering to the subject an effective amount of a compound of formula (I): 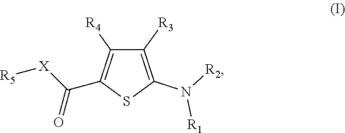
in which,
R₁ is H or C₁₋₆ alkyl;
R₂ is H, C₁₋₆ alkyl, or COR_{b}, R_{b} being C₁₋₆ alkyl;
R₃ is CN or CORₐ, Rₐ being OH, NH₂, NHCH₂CN, C₁₋₆ alkyl, or C₁₋₆ alkoxy;
R₄ is H, halo, C₁₋₆ alkyl, or C₁₋₆ alkoxy;
R₅ is aryl, heteroaryl, or C₁₋₆ alkyl; and
X is O, NH, or CH₂.

2. The method of claim 1, wherein R₁ is H and R₂ is H or C(O)CH₃.

3. The method of claim 1, wherein R₃ is CN, CO₂CH₃, CO₂CH₂CH₃, CO₂C(CH₃)₃, or COCH₃.

4. The method of claim 1, wherein R₄ is methyl.

5. The method of claim 1, wherein R₅ is aryl or heteroaryl.

6. The method of claim 5, wherein R₅ is selected from the group consisting of:

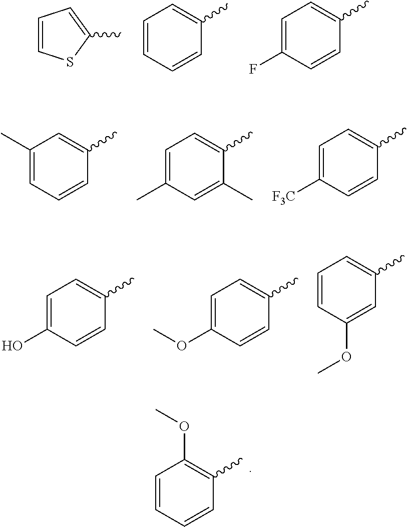

7. The method of claim 2, wherein R₃ is CN, CO₂CH₃, CO₂CH₂CH₃, CO₂C(CH₃)₃, or COCH₃; R₄ is methyl; and R₅ is aryl or heteroaryl.

8. The method of claim 1, wherein the compound is selected from the group consisting of Compounds 1-35.

9. The method of claim 8, wherein the compound is selected from the groups consisting of Compounds 1, 5, 6, 9, 10, 13, 16, 19, 20, 21, 25, 30, and 32.

10. The method of claim 1, wherein the Cisd2 insufficiency-associated disorder is non-alcoholic fatty liver disease, non-alcoholic steatohepatitis, chemotherapy-induced cardiotoxicity, chemotherapy-induced cachexia, hepatotoxicity, hypertensive cardiomyopathy, aging-related arrhythmogenesis, or an aging-related cardiac pathological change.

11. A method of increasing Cisd2 gene expression, the method comprising:
identifying a subject in need of increase in Cisd2 gene expression, and
administering to the subject an effective amount of a compound of formula (I): 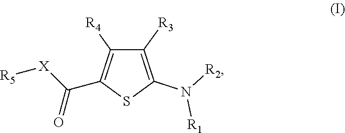
in which,
R₁ is H or C₁₋₆ alkyl;
R₂ is H, C₁₋₆ alkyl, or COR_{b}, R_{b} being C₁₋₆ alkyl;
R₃ is CN or CORₐ, Rₐ being OH, NH₂, NHCH₂CN, C₁₋₆ alkyl, or C₁₋₆ alkoxy;
R₄ is H, halo, C₁₋₆ alkyl, or C₁₋₆ alkoxy;
R₅ is aryl, heteroaryl, or C₁₋₆ alkyl; and
X is O, NH, or CH₂.

12. The method of claim 11, wherein R₁ is H and R₂ is H or C(O)CH₃.

13. The method of claim 11, wherein R₃ is CN, CO₂CH₃, CO₂CH₂CH₃, CO₂C(CH₃)₃, or COCH₃.

14. The method of claim 11, wherein R₄ is methyl.

15. The method of claim 11, wherein R₅ is aryl or heteroaryl.

16. The method of claim 15, wherein R₅ is selected from the group consisting of:

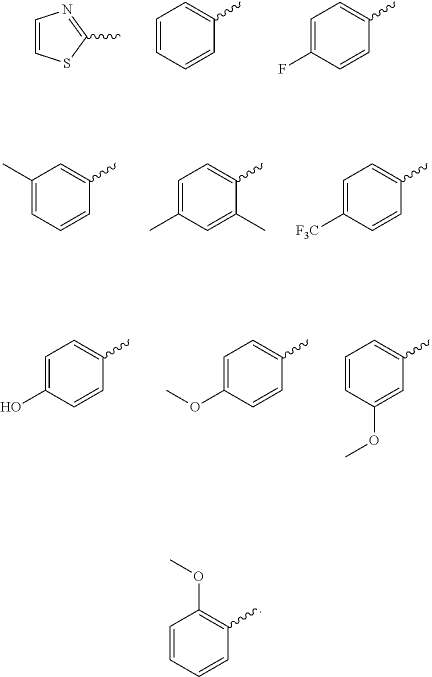

17. The method of claim 12, wherein R₃ is CN, CO₂CH₃, CO₂CH₂CH₃, CO₂C(CH₃)₃, or COCH₃; R₄ is methyl; and R₅ is aryl or heteroaryl.

18. The method of claim 11, wherein the compound is selected from the group consisting of Compounds 1-35.

19. The method of claim 18, wherein the compound is selected from the groups consisting of Compounds 1, 5, 6, 9, 10, 13, 16, 19, 20, 21, 25, 30, and 32.

20. The method of claim 11, wherein the subject suffers from a Cisd2 insufficiency-associated disorder selected from the group consisting of non-alcoholic fatty liver disease, non-alcoholic steatohepatitis, chemotherapy-induced cardiotoxicity, chemotherapy-induced cachexia, hepatotoxicity, hypertensive cardiomyopathy, aging-related arrhythmogenesis, and an aging-related cardiac pathological change.
